(19) United States
(12) Patent Application Publication
(10) Pub. No.: US 2023/0057979 A1
(43) Pub. Date: Feb. 23, 2023

(54) MEDICINAL COMPOSITIONS FOR CARBON DIOXIDE BASED METERED DOSE INHALERS

(71) Applicant: KINDEVA DRUG DELIVERY L.P., Woodbury, MN (US)

(72) Inventors: John P. Bunting, Castle Donington (GB); Sarah J. Dexter, Loughborough (GB); Peter D. Hodson, Breaston (GB); Alexander D. Slowey, Stapleford (GB)

(21) Appl. No.: 17/792,834

(22) PCT Filed: Jan. 12, 2021
(86) PCT No.: PCT/US2021/013055

§ 371 Date: Jul. 14, 2022

Related U.S. Application Data
(60) Provisional application No. 62/962,018, filed on Jan. 16, 2020.

(51) Int. Cl. A61K 9/00; A61K 31/137; A61K 31/56; A61K 31/573; A61K 45/06; A61K 47/02; A61K 9/12

(52) U.S. Cl. CPC A61K 9/008; A61K 31/137; A61K 31/56; A61K 31/573; A61K 45/06; A61K 47/02; A61K 9/12

(57) ABSTRACT

A pressurized medicinal composition comprising at least 40%, by weight, of liquid carbon dioxide, a fluid that forms a homogeneous solution with liquid carbon dioxide, and at least one active pharmaceutical ingredient dissolved or suspended in the composition. The composition may have a liquid to supercritical transition temperature greater than 40° C. Also, metered dose inhalers comprising an actuator, and a canister equipped with a metering valve, wherein the canister houses a reservoir comprising a pressurized carrier fluid mixture for dissolving or suspending at least one active pharmaceutical ingredient, the carrier fluid mixture comprising liquid carbon dioxide and a second component that is a liquid at room temperature and pressure.

### CROSS-REFERENCE TO RELATED APPLICATION

This application claims the benefit of U.S. Provisional Patent Application No. 62/962,018, filed Jan. 16, 2020, which is incorporated herein by reference in its entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or chronic obstructive pulmonary disease (COPD). pMDI devices can include an aluminum canister, sealed with a metering valve, that contains medicament formulation. Generally, the medicament formulation is a solution and/or suspension of one or more medicinal compounds in a liquefied hydrofluoroalkane (HFA) propellant.

In pulmonary pMDIs, the sealed canister can be provided to the patient in an actuator—a generally L-shaped plastic part comprising a generally vertical tube that surrounds the canister plus a generally horizontal tube that forms a patient portion (e.g., a mouthpiece or nosepiece) that can define an inspiration (or inhalation) orifice. The canister typically includes a metering valve that is crimped onto an appropriately sized metal can. The metal can is typically made of aluminium, having a wall thickness of approximately 0.5 mm. The canister contains a formulation typically comprising liquid propellant(s), drug(s), co-solvent(s) and excipient(s). To prevent loss of the formulation, (primarily the liquid propellant) the metering valve contains rubber components that form seals.

Historically, the propellants in most pMDIs had been chlorofluorocarbons (CFCs). However, due to stated environmental concerns during the 1990s led to the replacement of CFCs with hydrofluoroalkanes (HFAs) as the most commonly used propellant in pMDIs. Although HFAs do not cause ozone depletion they do have a stated high global warming potential (GWP), which is a measurement of the future radiative effect of an emission of a substance relative to that of the same amount of carbon dioxide (CO2). The two HFA propellants most commonly used in pMDIs are HFA134a (CF₃CH₂F) and HFA 227 (CF₃CHFCHF₃) having stated 100-year GWP values of 1300 to 1430 and 3220 to 3350, respectively.

Various other propellants have been proposed over the years. Among them, carbon dioxide (CO₂) has been mentioned as a potential propellant for pMDIs, but no pMDI product has been successfully developed and commercialized using carbon dioxide as a propellant.

### SUMMARY

It has now been found that despite CO₂'s major differences from other MDI propellants (such as much higher vapor pressure and different density, polarity, solubility, and component interaction characteristics) a practical pMDI can be made using CO₂. This can be very useful due to CO₂'s stated lower GWP (GWP value of 1).

In particular, in some embodiments, a pressurized medicinal composition includes about 40% to about 98%, by weight, of liquid carbon dioxide, about 2% to about 60%, by weight, of a second component that is a fluid that forms a solution with liquid carbon dioxide, and at least one active pharmaceutical ingredient dissolved or suspended in the composition. The presence of the second component (e.g., ethanol or isopropyl alcohol) can advantageously increase the liquid to supercritical phase transition temperature such that the inhaler remains stable over a normal range of use temperatures. Additionally, the second component (e.g., ethanol or isopropyl alcohol) can also lower the pressure within the canister, and/or increase the temperature of the aerosol spray delivered by an inhaler.

### BRIEF DESCRIPTION OF THE DRAWINGS

FIG. 1 is a side view of an inhaler including a canister containing a valve according to the present disclosure.

FIG. 2 is a plot of the supercritical point of CO₂ as a function of the amount of the ethanol or isopropyl alcohol as a second component of the medicinal composition.

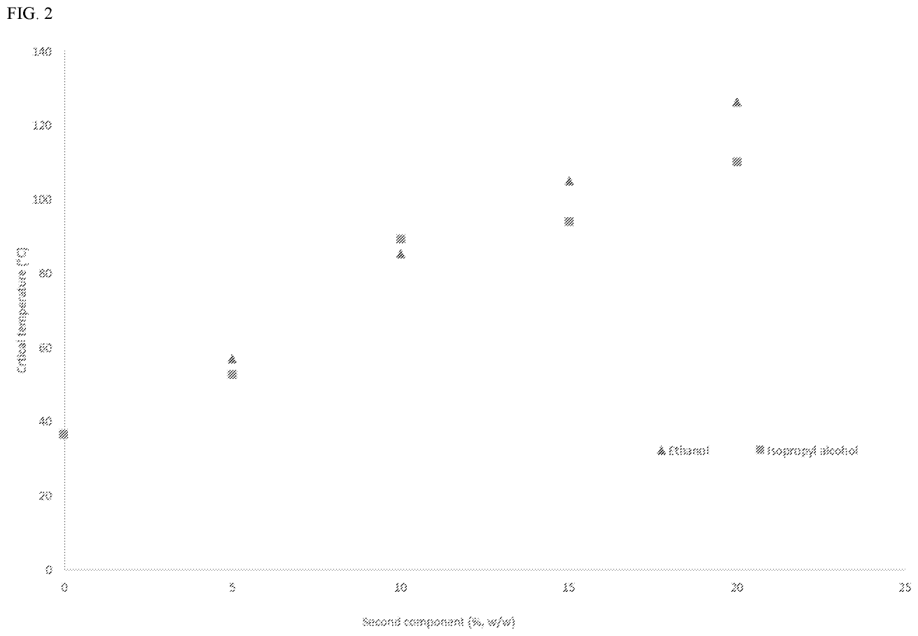

### DETAILED DESCRIPTION

Throughout this disclosure, singular forms such as “a,” “an,” and “the” are often used for convenience; singular forms are meant to include the plural unless the singular alone is explicitly specified or is clearly indicated by the context. Numerical ranges, for example “between x and y” or “from x to y”, include the endpoint values of x and y.

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and pMDIs, and methods of this disclosure; this terminology is not used to mean that these features are present, much less common, in the prior art. Unless otherwise specified, only the Background section of this Application refers to the prior art.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

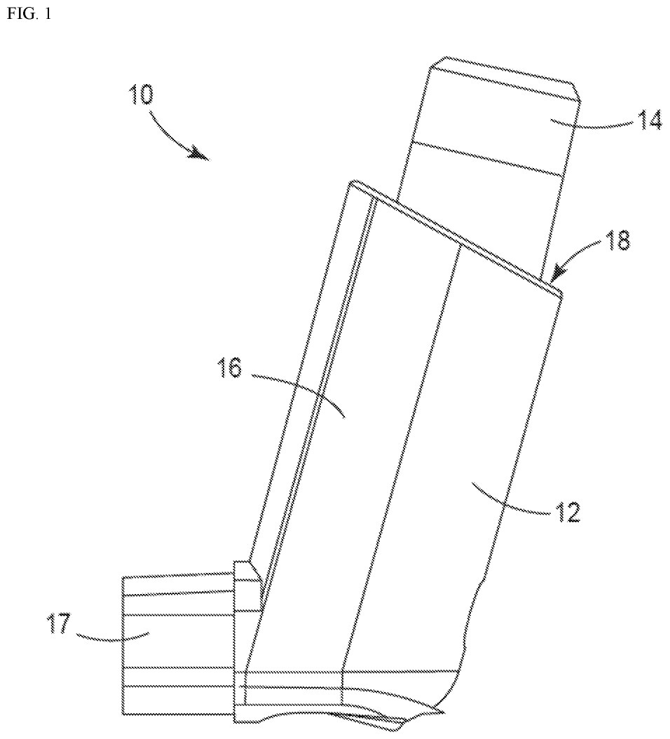

FIG. 1 depicts a pMDI 10 comprising an actuator 12 and a canister 14. Actuator 12 has a generally elongate actuator body 16 that acts as a housing for canister 14. Canister 14 is inserted into canister opening 18 at the top of actuator 12. Canister 14 is pressurized and contains a medicament formulation for delivery to a user via actuator 12 and mouthpiece 17 as will be described in further detail below. In other embodiments mouthpiece 17 can be replaced by a nosepiece (not depicted) to enable nasal delivery. In some embodiments, one or more active pharmaceutical ingredients (APIs) are dissolved and/or dispersed or suspended in the composition.

In some embodiments, the composition includes a carrier fluid mixture comprising a first component and a second component that form a homogeneous solution with the first component. The first component is liquid carbon dioxide. The second component is a fluid that may form a homogeneous solution with liquid carbon dioxide.

The carbon dioxide acts as a propellant to propel the composition from the canister 14 into the mouthpiece 17 and then to the patient. The carbon dioxide is present in the pressurized container as a liquid along with an amount of vapor phase carbon dioxide that is determined by the overall vapor pressure of the composition. As a propellant, the vapor pressure in equilibrium with liquid carbon dioxide at room temperature is sufficiently high that the gas phase may be expelled as an aerosol spray. In some embodiments, the concentration of carbon dioxide in the composition is about 40% to about 98%, by weight. In some embodiments, the concentration of carbon dioxide is about 50% to about 95%, about 60% to about 95%, about 70% to about 95%, or about 80% to about 90%, by weight. In some embodiments, the concentration of carbon dioxide is about 75%, about 80%, about 85%, about 90%, or about 95%, by weight. In some embodiments, carbon dioxide is the sole propellant in the composition.

In some embodiments, the concentration of carbon dioxide in the carrier fluid mixture is about 40% to about 98%, by weight. In some embodiments, the concentration of carbon dioxide in the carrier fluid mixture is about 50% to about 95%, about 60% to about 95%, about 70% to about 95%, or about 80% to about 90%, by weight. In some embodiments, the concentration of carbon dioxide in the carrier fluid mixture is about 75%, about 80%, about 85%, about 90%, or about 95%, by weight. In some embodiments, carbon dioxide is the sole propellant in the carrier fluid mixture.

Other propellants, such as hydrofluoroalkanes, including HFA-134a, HFA-227, or HFA-152, may be used as a minor component. Still other propellants include hydrofluoroolefins, including HFO-1234yf and HFO-1234ze. Amounts can include about 2% to about 20%, about 5% to about 20%, and about 5% to about 10%, by weight, of the composition.

In some embodiments, the composition may include a second component that is a liquid at room temperature, 23° C., and room pressure, 1 bar. Examples include polyethylene glycol having a molecular weight of 600 or less, ethanol, isopropyl alcohol, glycerol, water, or propylene glycol.

When the second component is included, and in particular when the second component is ethanol or isopropyl alcohol, the second component can be present in the composition at a minimum concentration of at least 2%, by weight, such as, for example, at least 5%, at least 10%, at least 15%, or at least 20%, by weight. When present, the second component can be present in the composition at a maximum concentration of no more than 60%, by weight, such as, for example, no more than 50%, no more than 40%, no more than 30%, no more than 20%, or no more than 10%, by weight. The second component is said to be present at a concentration “no more than” a reference concentration when the component is not absent but is present in an amount up to the reference concentration.

In some embodiments, the second component can be present in the composition at a concentration characterized as a range having endpoints defined by any minimum concentration set forth above and any maximum concentration set forth above that is greater than the minimum concentration. Thus, for example, the second component may be present in the composition at a concentration of from 2% to 60%, by weight, such as, for example, from 2% to 50%, from 2% to 40%, from 2% to 30%, from 2% to 20%, from 2% to 10%, from 5% to 50%, from 5% to 40%, from 5% to 30%, from 5% to 20% or from 5% to 10%, by weight.

In some embodiments, the second component may be present in the composition at a concentration that is equal to any minimum concentration or maximum concentration set forth above. Thus, the second component may be present in the composition at a concentration of 2%, 5%, 10%, 15%, 20%, 30%, 40%, 50%, or 60%, by weight.

The second component can form a solution with the liquid carbon dioxide. In some embodiments, the second component, and in particular ethanol or isopropyl alcohol, may aid in dissolving the active pharmaceutical ingredients (API). Where the second component dissolves the API(s) it may be desirable to initially mix the API with the second component to form a concentrate prior to adding propellant.

In some embodiments, the second component, and in particular ethanol or isopropyl alcohol, may aid in shifting the liquid to supercritical phase transition temperature. The liquid to supercritical phase transition temperature or critical point temperature for pure carbon dioxide is about 31° C. While this allows the carbon dioxide to be in the liquid phase (with associated vapor) at room temperature, it is possible that small increases in temperature will cause the carbon dioxide to transition to the supercritical state. The transition to the supercritical state causes a change in physical properties, including density and solubility. Thus, transition of carbon dioxide to the supercritical state may cause precipitation of an API or excipient that was in solution when the carbon dioxide was in its liquid phase. Conversely, an API or excipient that was suspended when the carbon dioxide was in its liquid phase may begin to Ostwald ripen and/or dissolve. Further, if the pMDI is used while the carbon dioxide is in the supercritical phase, then the weight of composition emitted from the metering valve will differ from the weight of composition that would be emitted from the metering valve when the carbon dioxide is in the liquid phase. This will cause an incorrect dose to be delivered.

The presence of the second component, and in particular ethanol or isopropyl alcohol, may increase the liquid-to-supercritical phase transition temperature to greater than or equal to about 40° C., about 50° C., or about 60° C. In some embodiments, a second component, and in particular ethanol or isopropyl alcohol, is added to the composition in an amount so that the liquid-to-supercritical phase transition temperature is from about 40° C. to about 180° C., from about 40° C. to about 150° C., from about 40° C. to about 120° C., or from about 50° C. to about 100° C.

In certain embodiments, a second component is added to the composition a in an amount so that the liquid-to-supercritical phase transition temperature is about 57° C., about 85° C., about 105° C., about 126° C., about 53° C., about 89° C., about 94° C., or about 110° C.

In some embodiments, the vapor pressure in equilibrium with the liquid phase of the second component is too low for it to be expelled as an aerosol spray. That is, the second component may be a non-propellant. The presence of the second component, in particular ethanol or isopropyl alcohol, may advantageously lower the pressure within the canister. At about 20° C. the vapor pressure of liquid carbon dioxide is about 57 bar. A second component, in particular ethanol or isopropyl alcohol, provided in amounts as described above may lower the vapor pressure of the composition at about 20° C. In some embodiments, the vapor pressure of the composition at about 20° C. is less than about 55 bar, less than about 53 bar, or less than about 50 bar. In some embodiments, the vapor pressure of the composition at about 20° C. is about 30 to about 55 bar, about 40 to about 55 bar, or about 45 to about 50 bar. In some embodiments, the vapor pressure of the composition at about 20° C. is about 46 bar, about 52 bar, or about 54 bar. At about 40° C. the vapor pressure of liquid carbon dioxide is about 80 bar. Adding a second component, in particular ethanol or isopropyl alcohol, in amounts as described above may lower the vapor pressure of the composition at about 40° C. In some embodiments, the vapor pressure of the composition at about 40° C. is less than about 75 bar, less than about 70 bar, or less than about 65 bar. In some embodiments, the vapor pressure of the composition at about 40° C. is about 40 to about 75 bar, about 40 to about 70 bar, or about 45 to about 65 bar. In some embodiments, the vapor pressure of the composition at about 40° C. is about 60 bar, about 65 bar, or about 70 bar.

The presence of a second component, in particular ethanol or isopropyl alcohol, may advantageously increase the temperature of the aerosol spray delivered by an inhaler. The spray temperatures of common propellants, such as HFA-152a and HFA-134a are well below about 0° C. and that of HFA-227 is about 2° C. In some embodiments, the spray temperature of the composition is greater than about 5° C., greater than about 8° C., or greater than about 12° C. In some embodiments, the spray temperature of the composition is about 8° C., about 10° C., or about 15° C.

The total amount of composition is desirably selected so that at least a portion of the carbon dioxide in the canister is present as a liquid after a predetermined number of medicinal doses have been delivered. The predetermined number of doses may be about 30 to about 200, about 60 to about 200, about 60 to about 120, about 60, about 120, about 200, or any other number of doses. The total amount of composition in the canister may be from about 1.0 to about 30.0 g, about 2.0 and about 20.0 g, about 5.0 and about 10.0 g. The total amount of composition is typically selected to be greater than the product of the predetermined number of doses times the metering volume of the metered valve. In some embodiments, the total amount of composition is greater than about 1.1, about 1.2, about 1.3, about 1.4, or about 1.5 times the product of the predetermined number of doses times the metering volume of the metered valve. This ensures that the amount of each dose remains relatively constant through the life of the inhaler.

The active pharmaceutical ingredient (API) may be a drug, vaccine, DNA fragment, hormone, other treatment, or a combination of any two APIs. Exemplary drugs can include those for the treatment of respiratory disorders, e.g., a bronchodilator, an anti-inflammatory (e.g., a corticosteroid), an anti-allergic, an anti-asthmatic, an antihistamine, or an anti-cholinergic agent. Thus, the API can include albuterol, terbutaline, ipratropium, oxitropium, tiotropium, TD 4208, beclomethasone, flunisolide, budesonide, mometasone, ciclesonide, cromolyn sodium, nedocromil sodium, ketotifen, azelastine, ergotamine, cyclosporine, aclidinium, umeclidinium, glycopyrrolate, salmeterol, fluticasone, formoterol, procaterol, indacaterol, carmoterol, milveterol, olodaterol, vilanterol, abediterol, omalizumab, zileuton, insulin, pentamidine, calcitonin, leuprolide, alpha-I-antitrypsin, interferon, triamcinolone, a pharmaceutically acceptable salt or ester of any of the listed drugs, or a mixture of any of the listed drugs, their pharmaceutically acceptable salts or their pharmaceutically acceptable esters. For fluticasone, exemplary esters include propionate or furoate; for beclomethasone, an exemplary ester is propionate; and for mometasone, an exemplary ester is furoate.

In some embodiments, the API(s) may be dissolved in the composition. In some embodiments, the API(s) may be dispersed or suspended in the composition. In the event a combination of two or more APIs are used, all the APIs may be suspended or in solution. Alternatively, one or more APIs may be suspended, while one or more APIs may be in solution. Where API is present in particulate form, i.e., suspended, it will generally have a mass median aerodynamic diameter in the range of about 1 to about 10 microns, preferably about 1 to about 5 microns.

The amount of API may be determined by the required dose per puff and the pMDI metering valve size, which may be from about 5 to about 200, about 25 and about 100, or about 25 and about 65 microliters. The concentration of each API is typically from about 0.01% to about 1.0%, by weight, sometimes from about 0.05% to about 0.5%, and as such, the medicament makes up a relatively small percentage of the total composition.

In use, the patient actuates the inhaler 10 by pressing downwardly on the canister 14. This moves the canister 14 into the body 18 of the actuator 12 and presses the canister valve stem against the actuator stem socket resulting in the canister metering valve opening and releasing a metered dose of composition that exits the mouthpiece 17 into the patient's mouth. It should be understood that other modes of actuation, such as breath-actuation, may be used as well and would operate as described with the exception that the force to depress the canister would be provided by the device, for instance by a spring or a motor-driven screw, in response to a triggering event, such as patient inhalation. Devices that may be used with medicament compositions of the present disclosure include those described in U.S. Pat. No. 6,032,836 (Hiscocks et al.), U.S. Pat. No. 9,010,329 (Hansen), U. K. Patent GB 2544128 B (Friel), and pending U. S. patent application (docket number 82728US002).

### EXAMPLES

### Composition Phase Behavior

The composition phase behavior was investigated by visual assessment using a stainless-steel pressure cell (SciMed, UK) fitted with two sapphire viewing windows (45 mm diameter, 10 mm thickness, secured in place by PEEK holders and sealed with silicone FEP encapsulated O-ring), a temperature probe (Type K, TC Direct, UK), and a pressure probe (Model S-20, Wika, UK) and four heater cartridges (50 W). The cell was filled at room temperature with liquid composition so that approximately 65% of the internal volume was occupied with composition. The composition was observed for evidence of a phase boundary between liquid and gas phase. The composition was agitated using a PTFE cross shaped stirrer bar (10 mm×5 mm, Thermo Fisher Scientific, Inc., Waltham, Mass.) at approximately 200 rpm using a magnetic stirrer. The composition was heated at a rate of approximately 5° C. per minute. The point at which the composition became supercritical was visually determined by the disappearance of the boundary between the liquid and gas phases. The critical temperature and pressure were recorded.

### Canister Filling/Inhaler Preparation

Formulations were prepared using a refillable two-part 12 g canister (Modern Combat Sports UK) equipped with a filling valve and an outlet valve. The canister was opened and a quantity of liquid containing the non-propellant components of the composition was dispensed into the open canister. The canister halves were attached to each other and the canister was filled through the filling valve with a quantity of carbon dioxide from a cylinder containing carbon dioxide using a needle charger (Modern Combat Sports, UK). The canister was shaken and then allowed to rest for approximately 15 minutes.

The outlet valve of the filled canister was attached to a sealed manifold equipped with a metering valve. The outlet valve was then fixed in the open position so that the canister and internal manifold volume formed a single pressurized reservoir volume. The metering valve was a type 20 DR 376/65/0 metering valve (Coster, Italy) modified by removing the mounting cup, internal gasket, external gasket, and spring. A PTFE O-ring (ID 2.57 mm×CS 1.78 mm, Polymax, UK) was added to provide the external seal around the metering valve stem. The canister-manifold-metering valve assembly was fitted with an actuator.

### Beclomethasone Dipropionate (BDP) Aerodynamic Particle Size Distribution (APSD) by NGI

APSD was determined using a Next Generation Impactor (NGI Model 170, Copley Scientific Limited UK) at a flow rate of 30±0.5 L/min drawn using a high-capacity pump (HCP5, Copley Scientific Limited, UK) the flow rate determined using a digital flow meter (Model 4043, TSI Instruments). The filter stage was fitted with a Whatman grade 934-AH glass microfiber filter. Units were tested at the start of life using a 3M Mark 6 actuator (3M, Loughborough, UK) with an exit orifice diameter of 0.25 mm and a jet length of 0.8 mm. Prior to testing, the inhaler was actuated five times to ensure the valve was primed after which the valve stem was cleaned and dried. The inhaler was then attached to the NGI with the use of an appropriate coupler and actuated five times into the NGI. The BDP was recovered from each component using methanol (HPLC grade, Thermo Fisher Scientific, Inc., Waltham, Mass.) using a volume of 10 mL for the stem/can, 30 mL for the throat/coupler, 5 mL for cups 1 to 4 and 20 mL for all other components. Recovery was performed using a rocker station (Gentle Rocker 4515, Copley Scientific Limited UK). Samples were assayed by reversed phase isocratic liquid chromatography using UV detection (Acquity H-Class UPLC, Waters Limited) with the parameter settings presented in Table 1.

TABLE 1
LC parameter settings for quantification of BDP

| | |
|---|---|
| Analytical Column | Acquity HSS C18, 2.1 × 50 mm, 1.8 μm |
| | (Waters Limited) |
| Mobile phase | Acetonitrile: water (60/40 v/v) (Both HPLC |
| | grade, Fisher Scientific) |
| Sample diluent | Methanol (HPLC grade, Fisher Scientific) |
| Detection wavelength | 238 nm |
| Detection resolution | 6 nm |
| Injection volume | 4 mcL |
| Flow rate | 0.75 ml/min |
| Column Temp | 25° C. |
| Run time 1.50 | 1.50 min |

### Salbutamol Sulphate Aerodynamic Particle Size Distribution (APSD) by NGI

APSD was determined using a Next Generation Impactor as described in the BDP APSD test method. Prior to testing, the inhaler was actuated five times to ensure the valve was primed after which the valve stem was cleaned and dried. The inhaler was then attached to the NGI with the use of an appropriate coupler and actuated six times into the NGI. The Salbutamol sulphate was recovered from each component using a solution of acetonitrile/0.1% phosphoric acid in water (9:1 v/v) using a volume of 20 mL for the throat/coupler and 10 mL for all other components. Recovery was performed using a rocker station (Gentle Rocker, Copley Scientific Limited UK). Samples were assayed by reversed phase gradient liquid chromatography using UV detection (HP1100, Agilent Technologies UK Limited), with the parameter settings in Table 2 and Table 3.

TABLE 2
LC parameter settings for quantification of Salbutamol Sulphate

| | |
|---|---|
| Analytical Column | KINETEX Core-Shell C18 (Phenomenex, Inc., |
| | Torrance, CA)), 150 mm × 4.6 mm, 5 μm |
| Detector | UV |
| Detection wavelength | 210 nm |
| Injection Volume | 30 mcL |
| Flow Rate | 1.0 mL/min |
| Column Temperature | 30° C. |
| Mobile Phase A | 0.1% phosphoric acid in water (both HPLC |
| | grade, Thermo Fisher Scientific, Inc., |
| | Waltham, MA) |
| Mobile Phase B | 0.1% phosphoric acid in acetonitrile (both |
| | HPLC grade, Thermo Fisher Scientific, Inc., |
| | Waltham, MA) |
| HPLC Run Time | 11 minutes |
| Diluent | acetonitrile: 0.1% phosphoric acid in water |
| | (9:1 v/v), (all HPLC grade, Thermo Fisher |
| | Scientific, Inc., Waltham, MA) |

TABLE 3
Liquid chromatography gradient profile
for Salbutamol Sulphate elution

| Time (min) | 0 | 2 | 2.5 | 5.5 | 6 | 11 |
|---|---|---|---|---|---|---|
| Mobile Phase A (%) | 93 | 93 | 81 | 81 | 93 | 93 |
| Mobile Phase B (%) | 7 | 7 | 19 | 19 | 7 | 7 |

### Spray Temperature

Temperature of the spray was determined using a plume temperature tester (Model PPT 1000, Copley Scientific Limited, UK) with no flowrate. Units were tested with a 3M Mark 6 actuator (3M, Loughborough, UK) having an exit orifice diameter of 0.25 mm and a jet length of 0.8 mm. The inhaler was actuated several times to ensure that the metering valve was primed. The outlet of the mouthpiece of an inhaler was placed 25 mm from and oriented perpendicular to the inlet of the plume temperature tester and the inhaler was actuated.

### Spray Force

The force of the spray was determined using a spray force tester (Model SFT 1000, Copley Scientific Limited, UK). Units were tested with a 3M Mark 6 actuator (3M, Loughborough, UK) having an exit orifice diameter of 0.25 mm and a jet length of 0.8 mm. The inhaler was actuated several times to ensure that the metering valve was primed. The outlet of the mouthpiece of an inhaler was placed 50 mm from and oriented perpendicular to the spray force tester and the inhaler was actuated.

### Through Life Shot Weight

A filled canister was prepared as described above and connected to an inhaler as described above. Units were tested with an aluminum actuator with a plastic insert having a 0.319 mm spray orifice. The inhaler was actuated with the plume directed towards waste collection. The weight of the inhaler was determined before and after each shot and the shot weight was determined from the difference. Results are reported as the number of shots for the shot weight to reach approximate steady state, the number of shots produced at steady state, and the average and standard deviation of the shot weights at steady state.

### Example 1

Approximately 8 g of a 2% (w/w) ethanol in carbon dioxide composition was prepared in a 12 g canister according to the canister filling procedure. The canister was allowed to rest for approximately 15 minutes. The canister was then connected to an inhaler device as described above. The spray force was determined as described above. Three replicate analyses were performed. The average spray force was 76 mN. The spray temperature was determined as described above. Three replicate analyses were performed. The average spray temperature was 6.3° C.

### Example 2

A 5% (w/w) ethanol in carbon dioxide composition was prepared in a pressure cell and tested according to the Composition Phase Behavior test method. The critical temperature was determined to be 57° C.

Approximately 8 g of a 5% (w/w) ethanol in carbon dioxide composition was prepared in a 12 g canister according to the canister filling procedure. The canister was allowed to rest for approximately 15 minutes. The canister was then connected to an inhaler device as described above. The spray force was determined as described above. Three replicate analyses were performed. The average spray force was 92 mN. The spray temperature was determined as described above. Three replicate analyses were performed. The average spray temperature was 4.7° C.

### Example 3

A 10% (w/w) ethanol in carbon dioxide composition was prepared in a pressure cell and tested as described in the Composition Phase Behavior test method. The critical temperature was determined to be 85° C.

Approximately 8 g of a 10% (w/w) ethanol in carbon dioxide composition was prepared in a 12 g canister according to the canister filling procedure. The canister was allowed to rest for approximately 15 minutes. The canister was then connected to an inhaler device as described above. The spray force was determined as described above. Three replicate analyses were performed. The average spray force was 65 mN. The spray temperature was determined as described above. Three replicate analyses were performed. The average spray temperature was 8.3° C.

Approximately 8.3 g of a 10% (w/w) ethanol in carbon dioxide composition was prepared in a 12 g canister according to the canister filling procedure. The canister was allowed to rest for approximately 15 minutes. The canister was then connected to an inhaler device as described above. The through life shot weights were determined as described above. After eleven shots the inhaler produced 78 shots with an average of 58.3+/−2.5 mg.

### Example 4

Approximately 8 g of a 20% (w/w) ethanol in carbon dioxide composition was prepared in a 12 g canister according to the canister filling procedure. The canister was allowed to rest for approximately 15 minutes. The canister was then connected to an inhaler device as described above. The spray force was determined as described above. Three replicate analyses were performed. The average spray force was 59 mN. The spray temperature was determined as described above. Three replicate analyses were performed. The average spray temperature was 9.3° C.

Approximately 8.2 g of a 20% (w/w) ethanol in carbon dioxide composition was prepared in a 12 g canister according to the canister filling procedure. The canister was allowed to rest for approximately 15 minutes. The canister was then connected to an inhaler device as described above. The through life shot weights were determined as described above. After three shots the inhaler produced 85 shots with an average of 62.0+/−0.5 mg.

### Example 5

Approximately 8 g of a 50% (w/w) ethanol in carbon dioxide composition was prepared in a 12 g canister according to the canister filling procedure. The canister was allowed to rest for approximately 15 minutes. The canister was then connected to an inhaler device as described above. The spray force was determined as described above. Three replicate analyses were performed. The average spray force was 35 mN. The spray temperature was determined as described above. Three replicate analyses were performed. The average spray temperature was 14.8° C.

### Example 6

A solution of beclomethasone dipropionate (Teva) in ethanol (100% BP/EP Hayman) was prepared by adding 0.60 g beclomethasone dipropionate to 10.00 g ethanol. A 0.81 g aliquot of the beclomethasone dipropionate in ethanol solution and 13.05 g of liquid carbon dioxide were combined in a pressure cell and tested as described in the Composition Phase Behavior test method. The composition was initially clear (i.e., free of particulate matter) and remained so during heating indicating that the beclomethasone dipropionate was in solution. The critical temperature was determined to be greater than 85° C. The pressure at 30° C. was 60 bar. The pressure at 40° C. was 68 bar.

A solution of beclomethasone dipropionate (Teva) in ethanol (99.5% Acros) was prepared by adding 0.23 g beclomethasone to 10 ml ethanol. A 0.87 g aliquot of the beclomethasone dipropionate in ethanol solution and 6.09 g of carbon dioxide were added to a refillable 12 g canister. The canister was shaken and then allowed to rest for approximately 15 minutes. The canister was connected to an inhaler device. The through life shot weights were determined. After three shots the inhaler produced 64 shots with an average of 64.5+/−0.5 mg.

A solution of beclomethasone dipropionate (Teva) in ethanol (100% BP/EP, Hayman) was prepared by adding 0.24 g beclomethasone to 16.00 g ethanol. Three 12 g canisters were each filled with an approximately 0.81 g aliquot of the beclomethasone dipropionate in ethanol solution and approximately 7.3 g of carbon dioxide. Each cylinder was allowed to rest for approximately 15 minutes. Each cylinder was connected to an inhaler device and the APSD determined as described above. The average aerodynamic particle size distribution is included in the table below.

TABLE 4
Beclomethasone dipropionate aerodynamic particle size distribution

| | | | Throat/ | | | | | | | | | |
|---|---|---|---|---|---|---|---|---|---|---|---|---|
| | Stem | Actuator | Coupler | Cup 1 | Cup 2 | Cup 3 | Cup 4 | Cup 5 | Cup 6 | Cup 7 | MOC | Filter |
| BDP | 3.6 | 10.8 | 59.0 | 0.3 | 0.1 | 0.1 | 0.8 | 4.3 | 7.4 | 4.7 | 5.9 | 1.6 |
| [mcg] | | | | | | | | | | | | |

### Example 7

A mixture of micronized salbutamol sulphate, d92<5 micron (Teva API, Israel) and ethanol (100% BP/EP Hayman, Essex, UK) was prepared by adding 0.19 g of salbutamol sulphate and mixing with 16.00 g of ethanol. The mixture was stirred to form a homogeneous suspension. Three 12 g canisters were each filled with an approximately 0.80 g aliquot of the salbutamol sulphate (SS) in ethanol suspension and approximately 7.3 g of carbon dioxide. Each cylinder was allowed to rest for approximately 15 minutes. Each cylinder was connected to an inhaler device and the APSD determined as described above. The average aerodynamic particle size distribution is included in the table below.

TABLE 5
Salbutamol sulphate aerodynamic particle size distribution

| | | | Throat/ | | | | | | | | | |
|---|---|---|---|---|---|---|---|---|---|---|---|---|
| | Stem | Actuator | Coupler | Cup 1 | Cup 2 | Cup 3 | Cup 4 | Cup 5 | Cup 6 | Cup 7 | MOC | Filter |
| SS | 1.4 | 7.9 | 54.2 | 0.20 | 0.07 | 0.45 | 2.5 | 3.7 | 2.1 | 0.60 | 0.29 | 0.05 |
| [mcg] | | | | | | | | | | | | |

### Example 8

A mixture of micronized salbutamol sulphate, d92<5 micron (Teva api, Israel) in ethanol (100% BP/EP Hayman, Essex, UK) was prepared by adding 0.29 g salbutamol to 7.5 g ethanol. The mixture was stirred to form a homogeneous suspension. A 0.79 g aliquot of the salbutamol in ethanol suspension and 14.83 g of liquid carbon dioxide were combined in a pressure cell and tested as described in the Composition Phase Behavior test method. The critical temperature was determined to be about 53° C. The pressure at 30° C. was 60 bar. The pressure at 40° C. was 69 bar.

### Example 9

A mixture of micronized fluticasone propionate (Hovione) and ethanol (100% BP/EP Hayman, Essex, UK) was prepared by adding 109 mg of fluticasone propionate to 3.95 g of ethanol. The mixture was stirred to form a homogeneous suspension. A 0.84 g aliquot of the fluticasone propionate and 6.4 g of carbon dioxide were added to a refillable 12 g canister. The canister was shaken and then allowed to rest for approximately 15 minutes. The canister was connected to the inhaler device. The through life shot weights were determined. After two shots the inhaler produced 62 shots with an average of 63.0+/−8.2 mg.

### Example 10

A mixture of micronized salbutamol sulphate (Teva API, Israel) and ethanol (100% BP/EP Hayman, Essex, UK) was prepared by adding 0.133 g of salbutamol sulphate and mixing with 7.89 g of ethanol. The mixture was stirred to form a homogeneous suspension. A 12 g canister was filled with a 0.846 g aliquot of the salbutamol sulphate (SS) in ethanol suspension, 0.862 g ethanol, and 7.142 g of carbon dioxide. The canister was shaken and then allowed to rest for approximately 15 minutes. The canister was connected to an inhaler device. The through life shot weights were determined. After three shots the inhaler produced 70 shots with an average of 64.3+/−2.7 mg.

### Example 11

A solution of beclomethasone dipropionate (Teva api, Israel) and formoterol fumarate dihydrate (Chemopharma, Vienna, Austria) in ethanol (100% BP/EP Hayman, Essex, UK) was prepared by adding 0.2566 g beclomethasone dipropionate and 0.0163 g formoterol fumarate dihydrate to 19.52 g ethanol. A 1.80 g aliquot of the beclomethasone dipropionate and formoterol fumarate dihydrate in ethanol solution and 15.71 g of liquid carbon dioxide were combined in a pressure cell as described in the Composition Phase Behavior test method. The composition was clear and colorless (i.e., free of particulate matter) and remained so for a duration of 2 hours indicating that the beclomethasone dipropionate and formoterol fumarate dihydrate were in solution.

### Example 12

A solution of beclomethasone dipropionate (Teva), formoterol fumarate dihydrate (Chemopharma, Vienna, Austria), and glycopyrronium bromide (Inke, Barcelona, Spain) in ethanol (100% BP/EP Hayman) was prepared by adding 0.3947 g beclomethasone dipropionate, 0.0253 g formoterol fumarate dihydrate, and 0.0659 g to 18.00 g ethanol. A 1.84 g aliquot of the beclomethasone dipropionate, formoterol fumarate dihydrate, and glycopyrronium bromide in ethanol solution and 15.53 g of liquid carbon dioxide were combined in a pressure cell as described in the Composition Phase Behavior test method. The composition was clear and colorless (i.e., free of particulate matter) indicating that the beclomethasone dipropionate, formoterol fumarate dihydrate, and glycopyrronium bromide were in solution.

### Example 13

A 15% (w/w) ethanol in carbon dioxide composition was prepared in a pressure cell and tested according to the Composition Phase Behavior test method. The critical temperature was determined to be 105° C.

### Example 14

A 20% (w/w) ethanol in carbon dioxide composition was prepared in a pressure cell and tested according to the Composition Phase Behavior test method. The critical temperature was determined to be 126° C.

### Example 15

A 5% (w/w) isopropyl alcohol in carbon dioxide composition was prepared in a pressure cell and tested according to the Composition Phase Behavior test method. The critical temperature was determined to be 53° C.

### Example 16

A 10% (w/w) isopropyl alcohol in carbon dioxide composition was prepared in a pressure cell and tested according to the Composition Phase Behavior test method. The critical temperature was determined to be 89° C.

### Example 17

A 15% (w/w) isopropyl alcohol in carbon dioxide composition was prepared in a pressure cell and tested according to the Composition Phase Behavior test method. The critical temperature was determined to be 94° C.

### Example 18

A 20% (w/w) isopropyl alcohol in carbon dioxide composition was prepared in a pressure cell and tested according to the Composition Phase Behavior test method. The critical temperature was determined to be 110° C.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. All references and publications cited herein are expressly incorporated herein by reference in their entirety into this disclosure. Various features and aspects of the present disclosure are set forth in the following claims.

1. A pressurized medicinal composition comprising:
about 40% to about 98%, by weight, of a first component that is liquid carbon dioxide,
about 2% to about 60%, by weight, of a second component that is a fluid that forms a solution with liquid carbon dioxide, and
at least one active pharmaceutical ingredient dissolved or suspended in the composition.

2. A pressurized medicinal composition comprising:
a carrier fluid and at least one active pharmaceutical ingredient dissolved or suspended in the carrier fluid,
wherein the carrier fluid comprises about 40% to about 98%, by weight, of a first component and about 2% to about 60%, by weight, of a second component mixed together in a homogeneous solution,
wherein the first component is liquid carbon dioxide, and
wherein the second component is liquid at room temperature and pressure.

3. A pressurized medicinal composition comprising:
a carrier fluid and at least one active pharmaceutical ingredient dissolved or suspended in the carrier fluid,
wherein the carrier fluid comprises about 40% to about 98%, by weight, of a first component and about 2% to about 60%, by weight, of a second component,
wherein the first component is a propellant,
wherein the second component is a non-propellant, and
wherein the first, propellant component is liquid carbon dioxide.

4. A pressurized medicinal composition comprising:
at least about 40%, by weight, of a first component that is liquid carbon dioxide,
an amount of a second component that is a fluid that forms a homogeneous solution with the liquid carbon dioxide, and
at least one active pharmaceutical ingredient dissolved or suspended in the composition,
wherein the liquid to supercritical transition temperature of the composition is greater than about 40° C.

5. A pressurized medicinal composition according to claim 1, wherein the first component is a propellant.

6. A pressurized medicinal composition according to claim 5, wherein carbon dioxide is the sole propellant.

7. A pressurized medicinal composition according to claim 1, wherein the liquid to supercritical transition temperature of the composition is greater than 40° C.

8. A pressurized medicinal composition according to claim 7 wherein the liquid to supercritical transition temperature of the composition is from about 40° C. to about 120° C.

9. A pressurized medicinal composition according to claim 1, wherein the second component is an alcohol.

10. A pressurized medicinal composition according to claim 9, wherein the second component is ethanol or isopropyl alcohol.

11. A pressurized medicinal composition according to claim 1, wherein the active pharmaceutical ingredient is selected from bronchodilators, corticosteroids, and anti-cholinergic agents.

12. A pressurized medicinal composition according to claim 1, wherein the amount of the second component is about 2% to about 20%, by weight.

13. A metered dose inhaler comprising:
a metering valve,
a canister, and
an actuator, wherein the canister contains a composition according to claim 1.

14. An inhaler according to claim 13, wherein the size of the metering valve is about 25 to about 100 microliters.

15. An inhaler according to claim 13, wherein the amount of composition in the canister is about 1 to about 30 mL.

16. An inhaler according to claim 13, wherein the canister contains a predetermined number of doses that is from about 30 to about 200.

17. An inhaler according to claim 13, wherein the pressure of the composition in the canister at 20° C. is from about 30 to about 55 bar.

18. An inhaler according to claim 13, wherein the pressure of the composition in the canister at 40° C. is from about 40 to about 75 bar.

19. An inhaler according to claim 13, wherein the spray temperature is greater than about 5° C.

20. A metered dose inhaler comprising an actuator, and a canister equipped with a metering valve, wherein the canister houses a reservoir comprising a pressurized carrier fluid mixture for dissolving or suspending at least one active pharmaceutical ingredient, the carrier fluid mixture comprising about 40% to about 98%, by weight, of a first component and about 2% to about 60%, by weight, of a second component mixed together in a homogeneous solution, wherein the first component is liquid carbon dioxide, and wherein the second component is liquid at room temperature and pressure.

21-34. (canceled)
